(19) United States
(12) Patent Application Publication
(10) Pub. No.: US 2009/0165379 A1
(43) Pub. Date: Jul. 2, 2009

(54) Coal Compositions for Catalytic Gasification

(75) Inventor: Alkis S. Rappas, Kingwood, TX (US)

Correspondence Address: MCDONNELL BOEHNEN HULBERT & BERGHOFF LLP, 300 S. WACKER DRIVE, SUITE 3100, CHICAGO, IL, 60606, US

(73) Assignee: GreatPoint Energy, Inc., Chicago, IL (US)

(21) Appl. No.: 12/342,578

(22) Filed: Dec. 23, 2008

Related U.S. Application Data
(60) Provisional application No. 61/017,300, filed on Dec. 28, 2007.

(51) Int. Cl. C07C 9/04

(52) U.S. Cl. 48/127.7; 44/504

(57) ABSTRACT

Particulate compositions are described comprising an intimate mixture of a coal and a gasification catalyst in the presence of steam to yield a plurality of gases including methane and at least one or more of hydrogen, carbon monoxide, carbon dioxide, hydrogen sulfide, ammonia and other higher hydrocarbons are formed. Processes are also provided for the preparation of the particulate compositions and converting the particulate composition into a plurality of gaseous products.

### CROSS-REFERENCE TO RELATED APPLICATIONS

This application claims priority under 35 U.S.C. §119 from U.S. Provisional Application Ser. No. 61/017,300 (filed Dec. 28, 2007), the disclosure of which is incorporated by reference herein for all purposes as if fully set forth.

### FIELD OF THE INVENTION

The present disclosure relates to particulate compositions of coal and at least one alkali metal and one transition metal gasification catalyst. Further, the disclosure relates to processes for preparation of the particulate compositions and for gasification of the same in the presence of steam to form gaseous products, and in particular, methane.

### BACKGROUND OF THE INVENTION

In view of numerous factors such as higher energy prices and environmental concerns, the production of value-added gaseous products from lower-fuel-value carbonaceous feedstocks, such as petroleum coke and coal, is receiving renewed attention. The catalytic gasification of such materials to produce methane and other value-added gases is disclosed, for example, in U.S. Pat. No. 3,828,474, U.S. Pat. No. 3,998,607, U.S. Pat. No. 4,057,512, U.S. Pat. No. 4,092,125, U.S. Pat. No. 4,094,650, U.S. Pat. No. 4,204,843, U.S. Pat. No. 4,468,231, U.S. Pat. No. 4,500,323, U.S. Pat. No. 4,541,841, U.S. Pat. No. 4,551,155, U.S. Pat. No. 4,558,027, U.S. Pat. No. 4,606,105, U.S. Pat. No. 4,617,027, U.S. Pat. No. 4,609,456, U.S. Pat. No. 5,017,282, U.S. Pat. No. 5,055,181, U.S. Pat. No. 6,187,465, U.S. Pat. No. 6,790,430, U.S. Pat. No. 6,894,183, U.S. Pat. No. 6,955,695, US2003/0167961A1, US2006/0265953A1, US2007/000177A1, US2007/083072A1, US2007/0277437A1 and GB1599932.

While it has been suggested to improve the gasification of coal by admixing coal with a selected catalyst, or catalysts, techniques heretofore suggested have not been entirely successful. For example, known methods of impregnating coal with catalyst include: a) physical admixing of catalyst with coal, and b) incipient wetness (“IW”) impregnation, wherein a catalyst-containing solution is added to a dry coat, and the volume of the solution is not in excess, but is instead just enough to completely fill the pores of the coal. These methods of coal impregnation suffer the drawback of producing a coal with catalyst loading that is not highly dispersed, and thus a coal with reduced gasification efficiency. The art has placed little emphasis on catalyst-loaded coal with highly dispersed catalyst loading, and processes to prepare same. Accordingly, a need exists in the art for providing new catalyst compositions to increase the yield of combustible gaseous products from catalytic coal gasification. In particular, a need exists in the art for providing new catalyst composition to increase the yield of methane from coal gasification.

### SUMMARY OF THE INVENTION

In a first aspect, the present invention provides a particulate composition comprising an intimate mixture of (a) a coal particulate having a size distribution suitable for gasification in a fluidized bed zone; (b) a transition metal gasification catalyst; (c) an alkaline earth metal source; and (d) an alkali metal gasification catalyst, wherein:

(i) in the presence of steam and under suitable temperature and pressure, the catalysts exhibit gasification activity whereby a plurality of gases including methane and at least one or more of hydrogen, carbon monoxide, carbon dioxide, hydrogen sulfide, ammonia and other higher hydrocarbons are formed;

(ii) the transition metal gasification catalyst is present in an amount sufficient to provide, in the particulate composition, a ratio of transition metal atoms to carbon atoms ranging from about 0.001 to about 0.10;

(iii) the alkaline earth metal source is present in an amount sufficient to provide, in the particulate composition, from about 0.1 to about 3.0 wt. % alkaline earth metal atoms, based on the total weight of the particulate composition on a dry basis; and

(iv) the alkali metal gasification catalyst is present in an amount sufficient to provide, in the particulate composition, a ratio of alkali metal atoms to carbon atoms ranging from about 0.01 to about 0.80.

In a second aspect, the invention provides a process for converting a particulate composition into a plurality of gaseous products, comprising the steps of: (a) supplying a particulate composition according the first aspect to a gasifying reactor; (b) reacting the particulate composition in the gasifying reactor in the presence of steam and under suitable temperature and pressure to form a plurality of gaseous including methane and at least one or more of hydrogen, carbon monoxide, carbon dioxide, hydrogen sulfide, ammonia and other higher hydrocarbons; and (c) at least partially separating the plurality of gaseous products to produce a stream comprising a predominant amount of one of the gaseous products.

In a third aspect, the invention provides a process for preparing a particulate composition comprising: (a) providing a particulate of a coal feedstock; (b) contacting the particulate with an aqueous solution comprising a transition metal source to form a first slurry; (c) dewatering the first slurry to form a first wet cake; (d) contacting the first wet cake with an aqueous solution comprising an alkaline earth metal source to form a second slurry; (e) dewatering the second slurry to form a second wet cake; (f) contacting the second wet cake with an aqueous solution comprising an alkali metal source to provide a third slurry; (g) dewatering the third slurry to form a third wet cake; and (h) drying the third wet cake to provide a particulate composition having a residual moisture content of about 6 wt % or less.

In a fourth aspect, the invention provides the particulate composition prepared according to the third aspect.

### DETAILED DESCRIPTION

The present disclosure relates to a particulate composition, methods for the preparation of the particulate composition, and methods for the catalytic gasification of the particulate composition. The methods of the present disclosure provide novel coal particulate compositions which comprise finely dispersed transition metal catalysts. The processes allow for the generation of such dispersed transition metal catalysts within the pores of the coal particulate, thereby enabling greater catalyst gasification activity and increased production of desired product gases (e.g, methane). Generally, the particulate composition includes various blends of, for example, high ash and/or high moisture content coals, particularly low ranking coals such as lignites, sub-bituminous coals, and mixtures thereof. Such particulate compositions can provide for an economical and commercially practical process for catalytic gasification of coals, such as lignites or sub-bituminous coal, with high ash and moisture contents to yield methane and other value-added gases as a product.

The present invention can be practiced, for example, using any of the developments to catalytic gasification technology disclosed in commonly owned US2007/0000177A1, US2007/0083072A1 and US2007/0277437A1; and U.S. patent application Ser. No. 12/178,380 (filed 23 Jul. 2008), Ser. No. 12/234,012 (filed 19 Sep. 2008) and Ser. No. 12/234,018 (filed 19 Sep. 2008). Moreover, the processes of the present invention can be practiced in conjunction with the subject matter of the following U.S. patent applications, each of which was filed on even date herewith: Ser. No. ______, entitled “PETROLEUM COKE COMPOSITIONS FOR CATALYTIC GASIFICATION” (attorney docket no. FN-0008 US NP1); Ser. No. ______, entitled “CATALYTIC GASIFICATION PROCESS WITH RECOVERY OF ALKALI METAL FROM CHAR” (attorney docket no. FN-0007 US NP1); Ser. No. ______, entitled “PETROLEUM COKE COMPOSITIONS FOR CATALYTIC GASIFICATION” (attorney docket no. FN-0011 US NP1); Ser. No. ______, entitled “CARBONACEOUS FUELS AND PROCESSES FOR MAKING AND USING THEM” (attorney docket no. FN-0013 US NP1); Ser. No. ______, entitled “CATALYTIC GASIFICATION PROCESS WITH RECOVERY OF ALKALI METAL FROM CHAR” (attorney docket no. FN-0014 US NP1); Ser. No. ______, entitled “STEAM GENERATING SLURRY GASIFIER FOR THE CATALYTIC GASIFICATION OF A CARBONACEOUS FEEDSTOCK” (attorney docket no. FN-0017 US NP1); Ser. No. ______, entitled “PROCESSES FOR MAKING SYNTHESIS GAS AND SYNGAS-DERIVED PRODUCTS” (attorney docket no. FN-0010 US NP1); Ser. No. ______, entitled “CATALYTIC GASIFICATION PROCESS WITH RECOVERY OF ALKALI METAL FROM CHAR” (attorney docket no. FN-0015 US NP1); Ser. No. ______, entitled “CATALYTIC GASIFICATION PROCESS WITH RECOVERY OF ALKALI METAL FROM CHAR” (attorney docket no. FN-0016 US NP1); Ser. No. ______, entitled “CONTINUOUS PROCESSES FOR CONVERTING CARBONACEOUS FEEDSTOCK INTO GASEOUS PRODUCTS” (attorney docket no. FN-0018 US NP1); and Ser. No. ______, entitled “PROCESSES FOR MAKING SYNGAS-DERIVED PRODUCTS” (attorney docket no. FN-0012 US NP1). All of the above are incorporated herein by reference for all purposes as if fully set forth.

All publications, patent applications, patents and other references mentioned herein, if not otherwise indicated, are explicitly incorporated by reference herein in their entirety for all purposes as if fully set forth.

Unless otherwise defined, all technical and scientific terms used herein have the same meaning as commonly understood by one of ordinary skill in the art to which this disclosure belongs. In case of conflict, the present specification, including definitions, will control.

Except where expressly noted, trademarks are shown in upper case.

Although methods and materials similar or equivalent to those described herein can be used in the practice or testing of the present disclosure, suitable methods and materials are described herein.

Unless stated otherwise, all percentages, parts, ratios, etc., are by weight.

When an amount, concentration, or other value or parameter is given as a range, or a list of upper and lower values, this is to be understood as specifically disclosing all ranges formed from any pair of any upper and lower range limits, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

When the term “about” is used in describing a value or an end-point of a range, the disclosure should be understood to include the specific value or end-point referred to.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but can include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The use of “a” or “an” to describe the various elements and components herein is merely for convenience and to give a general sense of the disclosure. This description should be read to include one or at least one and the singular also includes the plural unless it is obvious that it is meant otherwise.

The materials, methods, and examples herein are illustrative only and, except as specifically stated, are not intended to be limiting.

### Coal

The term “coal” as used herein means peat, lignite, sub-bituminous coal, bituminous coal, anthracite, or mixtures thereof. In certain embodiments, the coal has a carbon content of less than about 90%, less than about 85%, or less than about 80%, or less than about 75%, or less than about 70%, or less than about 65%, or less than about 60%, or less than about 55%, or less than about 50% by weight, based on the total coal weight. In other embodiments, the coal has a carbon content ranging up to about 90%, or up to about 85%, or up to about 80%, or up to about 75% by weight, based on the total coal weight. Examples of useful coals include, but are not limited to, Illinois #6, Pittsburgh #8, Beulah (ND), Utah Blind Canyon, and Powder River Basin (PRB) coals. Anthracite, bituminous coal, sub-bituminous coal, and lignite coal may contain about 10 wt %, from about 5 to about 7 wt %, from about 4 to about 8 wt %, and from about 9 to about 11 wt %, ash by total weight of the coal on a dry basis, respectively. However, the ash content of any particular coal source will depend on the rank and source of the coal, as is familiar to those skilled in the art. See, for example, “Coal Data: A Reference”, Energy Information Administration, Office of Coal, Nuclear, Electric and Alternate Fuels, U.S. Department of Energy, DOE/EIA-0064(93), February 1995.

### Catalyst Components

Particulate compositions according to the present disclosure are based on the above-described coal and further comprise an amount of an alkali metal gasification catalyst, a transition metal gasification catalyst, and an alkaline earth metal source.

The alkali metal gasification catalyst can be an alkali metal and/or a compound containing alkali metal atoms. For example, alkali metal gasification catalyst can comprise one or more alkali metal complexes (e.g., coordination complexes formed with one or more reactive functionalities on the surface or within the pores of the coal particulate, such as carboxylic acids and/or phenolic groups) formed with the coal particulate.

Typically, the quantity of the alkali metal component in the composition is sufficient to provide, in the particulate composition, a ratio of alkali metal atoms to carbon atoms ranging from about 0.01, or from about 0.02, or from about 0.03, or from about 0.04, to about 0.08, or to about 0.07, or to about 0.06.

The alkali metal component is typically loaded onto a coal particulate to achieve an alkali metal content of from about 3 to about 10 times more than the coal ash content, on a mass basis.

Suitable alkali metals include lithium, sodium, potassium, rubidium, cesium, and mixtures thereof. Particularly useful are potassium sources. Suitable alkali metal sources include alkali metal carbonates, bicarbonates, formates, oxalates, amides, hydroxides, acetates, or similar compounds. For example, the catalyst can comprise one or more of sodium carbonate, potassium carbonate, rubidium carbonate, lithium carbonate, cesium carbonate, sodium hydroxide, potassium hydroxide, rubidium hydroxide or cesium hydroxide, and particularly, one or more potassium complexes formed with the coal particulate, potassium carbonate, potassium bicarbonate, potassium hydroxide, or mixtures thereof.

The alkaline earth metal source can be an alkaline earth metal and/or a compound containing alkaline earth metal atoms. Typical alkaline earth metal sources can include magnesium, calcium, and/or barium sources, such as, but not limited to, magnesium oxide, magnesium hydroxide, magnesium carbonate, magnesium sulfate, calcium oxide, calcium hydroxide, calcium carbonate, calcium sulfate, barium oxide, barium hydroxide, barium carbonate, barium sulfate, or mixtures thereof. In certain embodiments, the alkaline earth source comprises a source of calcium; in certain other embodiments, the source of calcium is calcium hydroxide, calcium sulfate, or mixtures thereof.

Typically, the quantity of alkaline earth metal source in the composition is sufficient to provide from about 0.1 to about 3.0 wt %, or to about 2.0 wt %, alkaline earth atoms by dry weight.

The transition metal gasification catalyst can be a transition metal and/or a compound containing transition metal atoms. Typical transition metal gasification catalysts can include sources, such as, but not limited to, V, Cr, Mn, Fe, Co, Ni, Cu, Mo, or mixtures thereof. For example, transition metal gasification catalyst can comprise one or more transition metal complexes (e.g., coordination complexes formed with one or more reactive functionalities on the surface or within the pores of the coal particulate, such as carboxylic acids and/or phenolic groups). In certain embodiments, the transition metal gasification catalyst comprises an Fe source, Mn source, or mixtures thereof. In certain other embodiments, the transition metal gasification catalyst comprises one or more iron or manganese complexes formed with the coal particulate, FeO, Fe₂O₃, FeSO₄, MnO, MnO₂, MnSO₄, or mixtures thereof.

Typically, the quantity of transition metal gasification catalyst in the composition is sufficient to provide a ratio of transition metal atoms to carbon atoms ranging from 0.001 to about 0.10.

### Particulate Composition

Typically, the coal source for preparation of the particulate compositions can be supplied as a fine particulate having an average particle size of from about 25 microns, or from about 45 microns, up to about 2500 microns, or up to about 500 microns. One skilled in the art can readily determine the appropriate particle size for the individual particulates and the particulate composition. For example, when a fluid bed gasification reactor is used, the particulate composition can have an average particle size which enables incipient fluidization of the particulate composition at the gas velocity used in the fluid bed gasification reactor.

The particulate composition can comprise a blend of particulates from two or more sources. The ratio of the coal particulates in the particulate composition can be selected based on technical considerations, processing economics, availability, and proximity of the coal sources. The availability and proximity of the sources for these blends affect the price of the feeds, and thus the overall production costs of the catalytic gasification process. For example, an anthracite coal particulate and a sub-bituminous or lignite coal particulate can be blended in at about 5:95, about 10:90, about 15:85, about 20:80, about 25:75, about 30:70, about 35:65, about 40:60, about 45:55, about 50:50, about 55:45, about 60:40, about 65:35, about 70:20, about 75:25, about 80:20, about 85:15, about 90:10, or about 95:5 by weight on a wet or dry basis, depending on the processing conditions.

More significantly, the coal sources as well as the ratio the various coal particulates can be used to control other materials characteristics of the feedstock blend. Typically, coal includes significant quantities of inorganic mater including calcium and aluminum which form inorganic oxides (“ash”) in the gasification reactor. At temperatures above about 500 to 600° C., potassium and other alkali metals can react with ash to form insoluble alkali aluminosilicates. In this form, the alkali metal is inactive as a catalyst. To prevent buildup of the inorganic residue in a gasification reactor, a solid purge of char, i.e., solids composed of ash, unreacted carbonaceous material, and alkali metal bound within the solids, are routinely withdrawn. Catalyst loss in the solid purge is generally compensated by a substantial catalyst make-up stream.

The ash content of the particulate composition can be selected to be, for example, about 20 wt %, 15 wt %, or 10 wt % or lower, depending on ratio of the particulates and/or the starting ash in the various coal source. In other embodiments, the resulting particulate composition can comprise an ash content ranging from about 5 to about 25, from about 5 to about 20, from about 10 to about 20, or from about 10 to about 15, wt % based on the weight of the composition. In other embodiments, the ash content of the particulate composition can comprise less than about 15 wt %, 12 wt %, 10 wt %, 8 wt %, or 6 wt % alumina, based on the weight of the ash in the particulate composition. In certain embodiments, the resulting particulate composition can comprise an ash content of less than about 20 wt %, based on the weight of the particulate composition where the ash content of the particulate composition comprises less than about 15 wt % alumina, based on the weight of the ash in the particulate composition.

Such lower alumina values in the particulate composition allow for decreased losses of alkali catalysts in the gasification process. Typically, alumina can react with alkali source to yield an insoluble char comprising, for example, an alkali aluminate or aluminosilicate. Such insoluble char can lead to decreased catalyst recovery (i.e., increased catalyst loss), and thus, require additional costs of make-up catalyst in the overall gasification process, as will be discussed later.

Additionally, the resulting particulate composition can have a significantly higher % carbon, and thus btu/lb value and methane product per unit weight of the particulate composition. In certain embodiments, the resulting particulate composition has a carbon content ranging from about 75 wt %, or from about 80 wt %, or from about 85 wt %, or from about 90 wt %, up to about 95 wt %, based on the combined weight of the coal sources.

### Methods for Making the Particulate Composition

The coal sources for use in the preparation of the particulate composition can require initial processing to prepare the particulate composition for gasification. For example, when using a particulate composition comprising a mixture of two or more coal sources, each source can be separately processed to provide a particulate and add catalyst thereto, and subsequently mixed. In such a case, one coal source can be simply crushed into a particulate while the other is crushed and associated with the various gasification catalyst; the two particulates can subsequently be mixed.

The coal sources for the particulate composition can be crushed and/or ground separately according to any methods known in the art, such as impact crushing and wet or dry grinding to yield particulates of each. Depending on the method utilized for crushing and/or grinding of the coal sources, the resulting particulates can need to be sized (i.e., separated according to size) to provide an appropriate feedstock.

Any method known to those skilled in the art can be used to size the particulates. For example, sizing can be preformed by screening or passing the particulates through a screen or number of screens. Screening equipment can include grizzlies, bar screens, and wire mesh screens. Screens can be static or incorporate mechanisms to shake or vibrate the screen. Alternatively, classification can be used to separate the coal particulate. Classification equipment can include ore sorters, gas cyclones, hydrocyclones, rake classifiers, rotating trommels, or fluidized classifiers. The coal sources can be also sized or classified prior to grinding and/or crushing.

Additional feedstock processing steps can be necessary depending on the qualities of the coal sources. High-moisture coals can require drying prior to crushing. Some caking coals can require partial oxidation to simplify gasification reactor operation. Coal feedstocks deficient in ion-exchange sites can be pre-treated to create additional ion-exchange sites to facilitate catalysts loading and/or association. Such pre-treatments can be accomplished by any method known to the art that creates ion-exchange capable sites and/or enhances the porosity of the coal feed (see, for example, previously incorporated U.S. Pat. No. 4,468,231 and GB1599932). Often, pre-treatment is accomplished in an oxidative manner using any oxidant known to the art.

Typically, the coal is wet ground and sized (e.g., to a particle size distribution of 25 to 2500 microns) and then drained of its free water (i.e., dewatered) to a wet cake consistency. Examples of suitable methods for the wet grinding, sizing, and dewatering are known to those skilled in the art; for example, see previously incorporated U.S. patent application Ser. No. 12/178,380 (filed 23 Jul. 2008).

The filter cake of the coal particulate formed by the wet grinding in accordance with one embodiment of the present disclosure can have a moisture content ranging from about 40% to about 60%, about 40% to about 55%, or below 50%, based on the weight of the filter cake. It will be appreciated by one of ordinary skill in the art that the moisture content of dewatered wet ground coal depends on the particular type of coal, the particle size distribution, and the particular dewatering equipment used.

The coal particulate is subsequently treated, to associate a first catalyst, a second catalyst, and an alkaline earth source therewith, where the first and second catalysts are the alkali metal and transition metal gasification catalysts. The coal particulate can be treated in separate processing steps to provide the first catalyst and second catalysts as well as the alkaline earth source. For example, the transition metal gasification catalyst can be supplied to the coal particulate (e.g., a iron and/or manganese source), followed by a separate treatment to provide the alkaline earth metal source to the coal, followed by yet another separate treatment to provide the alkali metal gasification catalyst (e.g., potassium and/or sodium source) to the coal.

Any methods known to those skilled in the art can be used to associate one or more gasification catalysts and alkaline earth source with the coal particulate. Such methods include but are not limited to, admixing with a solid catalyst source and impregnating the catalyst on to coal particulate. Several impregnation methods known to those skilled in the art can be employed to incorporate the gasification catalysts. These methods include but are not limited to, incipient wetness impregnation, evaporative impregnation, vacuum impregnation, dip impregnation, ion exchanging, and combinations of these methods. Gasification catalysts can be impregnated into the coal particulate by slurrying with a solution (e.g., aqueous) of the catalyst.

When the coal particulate is slurried with a solution of the catalyst and/or co-catalyst, the resulting slurry can be dewatered to provide a particulate composition, again typically, as a wet cake. The catalyst solution for slurrying the coal particulate can be prepared from any catalyst source in the present methods, including fresh or make-up catalyst and recycled catalyst or catalyst solution (infra). Methods for dewatering the slurry to provide a wet cake of the catalyzed coal particulate include filtration (gravity or vacuum), centrifugation, vibratory screening, and/or a fluid press. Typically, when the coal particulate is treated, via slurrying with an aqueous solution, in separate steps to provide one or more of the transition metal gasification catalyst, alkali metal catalyst, and alkaline earth source, the slurry is dewatered between each treatment step.

In some cases, the slurry of the particulate with an aqueous solution of one of the gasification catalysts and/or alkaline earth source can be heated when contacting the catalyst therewith; such heating can occur at ambient or greater than ambient pressure and temperature.

One particular method suitable for combining the coal particulate with the gasification catalysts and alkaline earth source to provide a particulate composition where the various components have been associated with the coal particulate via ion exchange is described in previously incorporated U.S. patent application Ser. No. 12/178,380 (filed 23 Jul. 2008). The ion exchange loading mechanism is maximized (based on adsorption isotherms specifically developed for the coal), and the additional catalyst retained on wet including those inside the pores is controlled so that the total catalyst target value is obtained in a controlled manner. Such loading provides a particulate composition as a wet cake. The catalyst loaded and dewatered wet coal cake typically contains, for example, about 50% moisture. The total amount of catalyst loaded is controlled by controlling the concentration of catalyst components in the solution, as well as the contact time, temperature and method, as can be readily determined by those of ordinary skill in the relevant art based on the characteristics of the starting coal.

Ultimately, the wet cake of the coal particulate can be dried with a fluid bed slurry drier (i.e., treatment with superheated steam to vaporize the liquid), or the solution evaporated under reduced pressure, to provide a dry particulate composition.

The particulate composition of the invention typically comprises greater than about 50%, greater than about 70%, greater than about 85%, or greater than about 90% of the total amount of catalyst loaded associated with the coal matrix, for instance, as ion-exchanged catalyst on the acidic functional groups of the coal. The amount of each component associated with the coal particulate can be determined according to methods known to those skilled in the art.

As discussed previously, coal particulates from various sources can be combined appropriately to control, for example, the total catalyst loading and/or other qualities of the particulate composition. The appropriate ratios of the separate particulates will depend on the qualities of the feedstocks as well as the desired properties of the particulate composition. For example, coal particulates can be combined in such a ratio to yield a particulate composition having a predetermined ash content, as discussed previously.

The separate coal particulates can be combined by any methods known to those skilled in the art including, but not limited to, kneading, and vertical or horizontal mixers, for example, single or twin screw, ribbon, or drum mixers. The particulate composition can be stored for future use or transferred to a feed operation for introduction into a gasification reactor. The particulate composition can be conveyed to storage or feed operations according to any methods known to those skilled in the art, for example, a screw conveyer or pneumatic transport.

In one particularly useful method, a coal source is wet ground and dewatered to provide a first wet cake. The first wet cake is associated with a transition metal gasification catalyst (e.g., an iron or manganese source) via slurrying with an aqueous solution of the catalyst. The contacting of the first wet cake and the aqueous catalyst solution can occur at from about 25 to about 100, or from about 25 to about 75, or from about 50 to about 75° C. for a predetermined residence time. After contacting, the slurry is dewatered (e.g., via screening) to yield a second wet cake.

The second wet cake is contacted with an alkaline earth source (e.g., a calcium source) via slurrying with an aqueous solution of the source. Such slurrying can comprise contacting the particulate with one or more alkaline earth sources in the same or separate solutions. For example, the second wet cake can be slurried initially with a first alkaline earth source solution followed by treatment with a second alkaline earth source solution. The contacting of the second wet cake and the aqueous alkaline earth solution can occur at from about 25 to about 100, or from about 25 to about 75, or from about 50 to about 75° C. for a predetermined residence time. After completing the contacting, the slurry is dewatered (e.g., via screening) to yield a third wet cake.

Ultimately, the third wet cake is contacted with an alkali metal gasification catalyst (e.g., a potassium and/or sodium source) via methods familiar to those skilled in the art. For example, the third wet cake can be slurried with an aqueous solution of the alkali metal gasification catalyst. The contacting of the third wet cake and the aqueous alkali solution can occur at from about 100 to about 200, or from about 125 to about 175, or from about 140 to about 160° C. for a predetermined residence time. After contacting, the slurry is dewatered (e.g., via screening) to yield a fourth wet cake. This fourth wet cake is ultimately dried to yield a particulate composition having a water content of about 2, 4, or 6 wt % or less; or the fourth wet cake is ultimately dried to yield a particulate composition having a water content of from about 1 to about 6, from about 2 to about 6, from about 2 to about 5, or from about 2 to about 4 wt %.

Alternatively, the third wet cake can be contacted with an alkali metal gasification catalyst as a solid (and kneading together) to yield a fourth wet cake. This fourth wet cake is ultimately dried to yield a particulate composition having a water content of about 2, 4, or 6 wt % or less; or the fourth wet cake is ultimately dried to yield a particulate composition having a water content of from about 1 to about 6, from about 2 to about 6, from about 2 to about 5, or from about 2 to about 4 wt %.

In another alternative, the third wet cake can be contacted with an alkali metal gasification catalyst by adding (slurrying) a concentrated solution of the catalyst the third wet cake either before or during a drying process to yield a particulate composition having a water content of about 2, 4, or 6 wt % or less; or the particulate composition is dried to a water content of from about 1 to about 6, from about 2 to about 6, from about 2 to about 5, or from about 2 to about 4 wt %.

### Catalytic Gasification Methods

The particulate compositions of the present disclosure are particularly useful in integrated gasification processes for converting coal to combustible gases, such as methane. The gasification reactors for such processes are typically operated at moderately high pressures and temperature, requiring introduction of the particulate composition to the reaction zone of the gasification reactor while maintaining the required temperature, pressure, and flow rate of the feedstock. Those skilled in the art are familiar with feed systems for providing feedstocks to high pressure and/or temperature environments, including, star feeders, screw feeders, rotary pistons, and lock-hoppers. It should be understood that the feed system can include two or more pressure-balanced elements, such as lock hoppers, which would be used alternately.

In some instances, the particulate composition can be prepared at pressures conditions above the operating pressure of gasification reactor. Hence, the particulate composition can be directly passed into the gasification reactor without further pressurization.

Suitable gasification reactors include counter-current fixed bed, co-current fixed bed, fluidized bed, entrained flow, and moving bed reactors.

The particulate compositions are particularly useful for gasification at moderate temperatures of at least about 450° C., or of at least about 600° C. or above, to about 900° C., or to about 750° C., or to about 700° C.; and at pressures of at least about 50 psig, or at least about 200 psig, or at least about 400 psig, to about 1000 psig, or to about 700 psig, or to about 600 psig.

The gas utilized in the gasification reactor for pressurization and reactions of the particulate composition typically comprises steam, and optionally, oxygen or air, and are supplied to the reactor according to methods known to those skilled in the art. For example, any of the steam boilers known to those skilled in the art can supply steam to the reactor. Such boilers can be powered, for example, through the use of any carbonaceous material such as powdered coal, biomass etc., and including but not limited to rejected carbonaceous materials from the particulate composition preparation operation (e.g., fines). Steam can also be supplied from a second gasification reactor coupled to a combustion turbine where the exhaust from the reactor is thermally exchanged to a water source and produce steam.

Recycled steam from other process operations can also be used for supplying steam to the reactor. For example, when the slurried particulate composition is dried with a fluid bed slurry drier, as discussed previously, the steam generated through vaporization can be fed to the gasification reactor.

The small amount of required heat input for the catalytic coal gasification reaction can be provided by superheating a gas mixture of steam and recycle gas feeding the gasification reactor by any method known to one skilled in the art. In one method, compressed recycle gas of CO and H₂ can be mixed with steam and the resulting steam/recycle gas mixture can be further superheated by heat exchange with the gasification reactor effluent followed by superheating in a recycle gas furnace.

A methane reformer can be included in the process to supplement the recycle CO and H₂ fed to the reactor to ensure that the reaction is run under thermally neutral (adiabatic) conditions. In such instances, methane can be supplied for the reformer from the methane product, as described below.

Reaction of the particulate composition under the described conditions typically provides a crude product gas and a char. The char produced in the gasification reactor during the present processes typically is removed from the gasification reactor for sampling, purging, and/or catalyst recovery. Methods for removing char are well known to those skilled in the art. One such method taught by EP-A-0102828, for example, can be employed. The char can be periodically withdrawn from the gasification reactor through a lock hopper system, although other methods are known to those skilled in the art. Processes have been developed to recover alkali metal from the solid purge in order to reduce raw material costs and to minimize environmental impact of a CCG process.

The char can be quenched with recycle gas and water and directed to a catalyst recycling operation for extraction and reuse of the alkali metal catalyst. Particularly useful recovery and recycling processes are described in U.S. Pat. No. 4,459,138, as well as previously incorporated U.S. Pat. No. 4,057,512, US2007/0277437A1, U.S. patent application Ser. No. ______, entitled “CATALYTIC GASIFICATION PROCESS WITH RECOVERY OF ALKALI METAL FROM CHAR” (attorney docket no. FN-0007 US NP1), U.S. patent application Ser. No. ______, entitled “CATALYTIC GASIFICATION PROCESS WITH RECOVERY OF ALKALI METAL FROM CHAR” (attorney docket no. FN-00014 US NP1), U.S. patent application Ser. No. ______, entitled “CATALYTIC GASIFICATION PROCESS WITH RECOVERY OF ALKALI METAL FROM CHAR” (attorney docket no. FN-0015 US NP1), and U.S. patent application Ser. No. ______, entitled “CATALYTIC GASIFICATION PROCESS WITH RECOVERY OF ALKALI METAL FROM CHAR” (attorney docket no. FN-0016 US NP1). Reference can be had to those documents for further process details.

Crude product gas effluent leaving the gasification reactor can pass through a portion of the gasification reactor which serves as a disengagement zone where particles too heavy to be entrained by the gas leaving the gasification reactor (i.e., fines) are returned to the fluidized bed. The disengagement zone can include one or more internal cyclone separators or similar devices for removing fines and particulates from the gas. The gas effluent passing through the disengagement zone and leaving the gasification reactor generally contains CH₄, CO₂, H₂ and CO, H₂S, NH₃, unreacted steam, entrained fines, and other contaminants such as COS.

The gas stream from which the fines have been removed can then be passed through a heat exchanger to cool the gas and the recovered heat can be used to preheat recycle gas and generate high pressure steam. Residual entrained fines can also be removed by any suitable means such as external cyclone separators followed by Venturi scrubbers. The recovered fines can be processed to recover alkali metal catalyst.

The gas stream exiting the Venturi scrubbers can be fed to COS hydrolysis reactors for COS removal (sour process) and further cooled in a heat exchanger to recover residual heat prior to entering water scrubbers for ammonia recovery, yielding a scrubbed gas comprising at least H₂S, CO₂, CO, H₂, and CH₄. Methods for COS hydrolysis are known to those skilled in the art, for example, see U.S. Pat. No. 4,100,256.

The residual heat from the scrubbed gas can be used to generate low pressure steam. Scrubber water and sour process condensate can be processed to strip and recover H₂S, CO₂ and NH₃; such processes are well known to those skilled in the art. NH₃ can typically be recovered as an aqueous solution (e.g., 20 wt %).

A subsequent acid gas removal process can be used to remove H₂S and CO₂ from the scrubbed gas stream by a physical absorption method involving solvent treatment of the gas to give a cleaned gas stream. Such processes involve contacting the scrubbed gas with a solvent such as monoethanolamine, diethanolamine, methyldiethanolamine, diisopropylamine, diglycolamine, a solution of sodium salts of amino acids, methanol, hot potassium carbonate or the like. One method can involve the use of Selexol® (UOP LLC, Des Plaines, Ill. USA) or Rectisol® (Lurgi AG, Frankfurt am Main, Germany) solvent having two trains; each train consisting of an H₂S absorber and a CO₂ absorber. The spent solvent containing H₂S, CO₂ and other contaminants can be regenerated by any method known to those skilled in the art, including contacting the spent solvent with steam or other stripping gas to remove the contaminants or by passing the spent solvent through stripper columns. Recovered acid gases can be sent for sulfur recovery processing. The resulting cleaned gas stream contains mostly CH₄, H₂, and CO and, typically, small amounts of CO₂ and H₂O. Any recovered H₂S from the acid gas removal and sour water stripping can be converted to elemental sulfur by any method known to those skilled in the art, including the Claus process. Sulfur can be recovered as a molten liquid.

The cleaned gas stream can be further processed to separate and recover CH₄ by any suitable gas separation method known to those skilled in the art including, but not limited to, cryogenic distillation and the use of molecular sieves or ceramic membranes. One method for recovering CH₄ from the cleaned gas stream involves the combined use of molecular sieve absorbers to remove residual H₂0 and CO₂ and cryogenic distillation to fractionate and recover CH₄. Typically, two gas streams can be produced by the gas separation process, a methane product stream and a syngas stream (H₂ and CO). The syngas stream can be compressed and recycled to the gasification reactor. If necessary, a portion of the methane product can be directed to a reformer, as discussed previously and/or a portion of the methane product can be used as plant fuel.

### EXAMPLES

### Example 1

Lignite Particulate Composition

367 g of wet ground Powder River Basin coal (54.5 wt % moisture, 69.9 wt. % C, dry basis) is slurried with a soaking solution containing ferrous sulfate hepahydrate (24.37 g) and manganese (II) sulfate hydrate (11.22 g) in water (633 g). The slurry density is approximately 20 wt %. The slurry is maintained and stirred for 2 hours at 65° C. The treated coal is dewatered by filtering over a vibratory screen with a mesh size of about +325 to yield a wet coal cake. The wet cake is slurried with a soaking solution containing calcium hydroxide (3.34 g) and calcium sulfate dihydrate (1.44 g) in water (575 g). The slurry density is approximately 20 wt %. The slurry is maintained and stirred for 2 hours at 65° C. The treated coal is dewatered by filtering over a vibratory screen with a mesh size of about +325 to yield a second wet coal cake. The second wet coal cake is slurried with a soaking solution containing potassium hydroxide (9.43 g) and potassium carbonate (104.4 g) in water (575 g). The slurry density is approximately 20 wt %. The slurry is maintained and stirred for 2 hours at 150° C. The treated coal is dewatered by filtering over a vibratory screen with a mesh size of about +325 to yield a third wet coal cake. Finally, the third wet coal cake is dried to yield a particulate composition having about 2 wt % residual moisture.

We claim:

1. A particulate composition comprising an intimate mixture of (a) a coal particulate having a size distribution suitable for gasification in a fluidized bed zone; (b) a transition metal gasification catalyst; (c) an alkaline earth metal source; and (d) an alkali metal gasification catalyst, wherein:
(i) in the presence of steam and under suitable temperature and pressure, the catalysts exhibit gasification activity whereby a plurality of gases including methane and at least one or more of hydrogen, carbon monoxide, carbon dioxide, hydrogen sulfide, ammonia and other higher hydrocarbons are formed;
(ii) the transition metal gasification catalyst is present in an amount sufficient to provide, in the particulate composition, a ratio of transition metal atoms to carbon atoms ranging from 0.001 to about 0.10;
(iii) the alkaline earth metal source is present in an amount sufficient to provide, in the particulate composition, from about 0.1 to about 3.0 wt % alkaline earth metal atoms on a dry basis; and
(iv) the alkali metal gasification catalyst is present in an amount sufficient to provide, in the particulate composition, a ratio of alkali metal atoms to carbon atoms ranging from 0.01 to about 0.08.

2. The particulate composition according to claim 1, wherein the alkali metal gasification catalyst comprises potassium and/or sodium.

3. The particulate composition according to claim 1, wherein the alkaline earth metal source comprises a source of calcium, magnesium or barium.

4. The particulate composition according to claim 2, wherein the alkaline earth metal source comprises a source of calcium, magnesium or barium.

5. The particulate composition according to claim 1, wherein the transition metal gasification catalyst comprises V, Cr, Mn, Fe, Co, Ni, Cu, Mo, or mixtures thereof.

6. The particulate composition according to claim 1, wherein the transition metal gasification catalyst comprises Fe, Mn, or mixtures thereof.

7. The particulate composition according to claim 2, wherein the transition metal gasification catalyst comprises Fe, Mn, or mixtures thereof.

8. The particulate composition according to claim 4, wherein the transition metal gasification catalyst comprises Fe, Mn, or mixtures thereof.

9. The particulate composition according to claim 1, having a particle size ranging from about 25 microns to about 2500 microns.

10. The particulate composition according to claim 1, having a residual moisture content of less than about 6 wt %.

11. A process for converting a particulate composition into a plurality of gaseous products comprising the steps of:
(a) supplying a particulate composition according to claim 1 to a gasifying reactor;
(b) reacting the particulate composition in the gasifying reactor in the presence of steam and under suitable temperature and pressure to form a plurality of gaseous including methane and at least one or more of hydrogen, carbon monoxide, carbon dioxide, hydrogen sulfide, ammonia and other higher hydrocarbons; and
(c) at least partially separating the plurality of gaseous products to produce a stream comprising a predominant amount of one of the gaseous products.

12. The process according to claim 11, wherein the stream comprises a predominant amount of methane.

13. A process for preparing a particulate composition, comprising the steps of:
(a) providing a particulate of a coal feedstock;
(b) contacting the particulate with an aqueous solution comprising a transition metal source to form a first slurry;
(c) dewatering the first slurry to form a first wet cake;
(d) contacting the first wet cake with an aqueous solution comprising an alkaline earth metal source to form a second slurry;
(e) dewatering the second slurry to form a second wet cake;
(f) contacting the second wet cake with an alkali metal gasification catalyst to form a third wet cake; and
(g) drying the third wet cake to provide a particulate composition having a residual moisture content of less than about 6 wt %.

14. The process according to claim 13, wherein the contacting the second wet cake with an alkali metal gasification catalyst to form a third wet cake comprises the steps of:
(i) contacting the second wet cake with an aqueous solution comprising an alkali metal source to provide a third slurry; and
(ii) dewatering the third slurry to form the third wet cake.

15. The process according to claim 13, wherein the alkali metal gasification catalyst comprises potassium and/or sodium.

16. The process according to claim 13, wherein the alkaline earth metal source comprises a source of calcium, magnesium or barium.

17. The process according to claim 15, wherein the alkaline earth metal source comprises a source of calcium, magnesium or barium.

18. The process according to claim 13, wherein the transition metal gasification catalyst comprises Fe, Mn, or mixtures thereof.

19. The process according to claim 17, wherein the transition metal gasification catalyst comprises Fe, Mn, or mixtures thereof.
